(19) United States
(12) Patent Application Publication
(10) Pub. No.: US 2014/0303670 A1
(43) Pub. Date: Oct. 9, 2014

(54) Method and Device for Spinal Analysis

(71) Applicant: Neuromechanical Innovations, LLC, Chandler, AZ (US)

(72) Inventor: Christopher J. Colloca, Chandler, AZ (US)

(21) Appl. No.: 14/358,902

(22) PCT Filed: Nov. 16, 2012
(86) PCT No.: PCT/US2012/065720

§ 371 Date: May 16, 2014

Related U.S. Application Data
(60) Provisional application No. 61/560,727, filed on Nov. 16, 2011.
(60) Provisional application No. 61/560,732, filed on Nov. 16, 2011.

(51) Int. Cl. A61B 17/66

(52) U.S. Cl. CPC A61B 17/66
USPC 606/237

(57) ABSTRACT

A method for interpreting patient treatment results and data is described. The Method uses an electric chiropractic adjusting instrument and software program and preferably comprises the following steps of adjusting a spine of a patient using the electric chiropractic adjusting instrument; gathering electric chiropractic instrument adjustment thrust data, patient spine response to thrusts of the electric chiropractic adjustment instrument, and objective clinician findings; recording the electric chiropractic instrument adjustment thrust data, the patient spine response to the thrusts, and the objective clinician findings in a database; providing real time results and findings; and generating reports based on the thrust data, the patient spine response to the thrusts of the electric chiropractic adjustment instrument, and the objective clinician findings.

### FIELD

This invention relates to medical devices, software and methods related thereto. Preferably, the invention relates to chiropractic medical devices, software and methods related thereto. This patent claims benefit to U.S. Provisional Application Nos. 61/560,732 and 61/560,727 both filed Nov. 16, 2011 each of which is hereby incorporated by reference in their entireties.

### BACKGROUND

The purpose of the product is to provide a tool to assist primarily chiropractors and chiropractic clinical technicians (generic clinician), in the treatment of their patients. The name of the system should be Impulse DSA (Dynamical Spinal Analysis) System. While other programs may be later made, the name of the software program for description herein is Impulse DSA Program. While other devices and methods for those devices may be made within the scope of this invention, the device used herein is an electric chiropractic adjusting instrument also referred to as iQ Impulse instrument in one embodiment. Certain descriptions of a compatible devices may found in US Patent Publication No. US-2007-0150004-A1 on Jun. 28, 2007 (filed and U.S. patent application Ser. No. 11/567,007 on Dec. 5, 2006 now issued as U.S. Pat. No. 8,083,699) which is incorporated herein in its entirety. U.S. Pat. No. 8,083,699 relates to a method for controlling an electric chiropractic adjusting instrument comprising the steps of: Initializing data relating to a maximum spinal mobility; Resetting a peak maximum reading of an accelerometer peak signal; Resetting a detector circuit wherein the detector circuit transmits the data between an electrically driven impact head and a sensing device; Activating the electrically driven impact head to contact a body at least twice; Reading the accelerometer peak signal generated within the impact head using the sensing device; Repeating the step of activating the electrically driven impact head wherein a first reading and at least one subsequent reading are generated; Evaluating a plurality of sensing device readings using a sensing processing unit; Comparing the first reading and the multiple subsequent readings to determine if a maximum spinal mobility has been obtained; and Deactivating the electrically driven impact head when the first reading of the sensing device is exceeded by a subsequent reading of the sensing device such that the maximum spinal mobility has been obtained. U.S. Pat. No. 8,083,699 also relates to a method for setting a pulse rate of an electric chiropractic adjusting instrument comprising the steps of: Initializing data relating to a pulse rate of the electric chiropractic adjusting instrument; Resetting a peak maximum reading of an accelerometer peak signal; Resetting a detector circuit wherein the detector circuit transmits the data between an electrically driven impact head and a sensing device; Activating the electrically driven impact head to contact a body at least twice; Reading the accelerometer peak signal generated within the impact head using the sensing device; Repeating the step of activating the electrically driven impact head wherein a first reading and at least one subsequent reading are generated; Comparing the first reading and the multiple subsequent readings; and Evaluating a plurality of sensing device readings using a sensing processing unit; wherein the sensing processing unit is used to set the pulse rate. In addition, U.S. Pat. No. 8,083,699 relates to a method for setting a pulse rate of an electric chiropractic adjusting instrument comprising the steps of: Initializing data relating to a pulse rate of the electric chiropractic adjusting instrument; Resetting a peak maximum reading of an accelerometer peak signal; Resetting a detector circuit wherein the detector circuit transmits the data between an electrically driven impact head and a sensing device; Activating the electrically driven impact head to contact a body at least twice; Reading the accelerometer peak signal generated within the impact head using the sensing device; Repeating the step of activating the electrically driven impact head wherein a first reading and at least one subsequent reading are generated; Comparing the first reading and the multiple subsequent readings; Evaluating a plurality of sensing device readings using a sensing processing unit; wherein the sensing processing unit is used to set the pulse rate; and Controlling a dosage delivered wherein the dosage is an amount of times the step of activating the impact head to contact a body is performed.

The Impulse DSA System is intended to support the treatment of chiropractic patients by allowing the clinician to make selections for different skeletal levels for adjustment; by recording the iQ instrument adjustment thrust data, the patient's response to those thrusts, the subjective findings from the patient, and the objective finding from the clinician; and by generating reports for each patient visit. A number of graphs are produced for doctor analyses used for planning further treatment of the patient. The term “adjustment” as used herein is for the purposes of clinical analysis and treatment of the musculoskeletal system and includes manipulation, mobilization, manual therapy and chiropractic techniques.

### SUMMARY

One of the preferred inventions is a method for interpreting patient treatment results and data using an electric chiropractic adjusting instrument and software program is described and preferably comprises the following steps of adjusting a spine of a patient using the electric chiropractic adjusting instrument; gathering electric chiropractic instrument adjustment thrust data, patient spine response to thrusts of the electric chiropractic adjustment instrument, and objective clinician findings; recording the electric chiropractic instrument adjustment thrust data, the patient spine response to the thrusts, and the objective clinician findings in a database; providing real time results and findings; and generating reports based on the thrust data, the patient spine response to the thrusts of the electric chiropractic adjustment instrument, and the objective clinician findings. More preferably, method for interpreting patient treatment results and data further comprises one or more of the following: the step of displaying the real time results and findings on a graphical user interface or the step of analyzing and recording subjective findings from the patient; the step of producing graphs for clinician analysis to plan further treatment of the patient based on the results and findings; the step of storing the results and data on a remote server; and/or the step of storing the results and data on a local server.

Another of the preferred inventions is a method to modify an electric chiropractic adjusting instrument thrust adjustment using recorded patient data and preferably comprises comprising the steps of: gathering electric chiropractic instrument adjustment thrust data, patient spine response to thrusts of the electric chiropractic adjustment instrument, and objective clinician findings; recording the electric chiropractic instrument adjustment thrust data, the patient spine response to the thrusts, and the objective clinician findings in a database; providing real time results and findings to a clinician based on the gathered data; and modifying the thrust adjustment of the electric chiropractic instrument based on the adjustment thrust data, the patient spine response to the thrusts, and the objective clinician findings. More preferably, method for interpreting patient treatment results and data further comprises one or more of the following: the step of displaying the real time results on a graphical user interface; the step of analyzing and recording subjective findings from the patient; and/or the step of producing graphs for clinician analysis to modify the electric chiropractic adjusting instrument based on the results and findings

### BRIEF DESCRIPTION OF THE DRAWINGS

FIG. 1 shows a block functional diagram of a preferred embodiment of the invention sometimes referred to Impulse DSA system herein.

### DESCRIPTION OF PREFERRED EMBODIMENTS

Preferably, but not required or limited to, the Impulse DSA System provides the following functions:

1. iQ Instrument Function

2. Home Function

3. EHR (Electronic Health Record) Function

4. Patient Information Function

5. Health History Function

6. Subjective Function

7. Adjust Function

8. Review Function

9. Results Function

10. Educate Function

11. Assessment Function

12. Plan Function

13. Outcome Assessments Function

14. E/M Function

15. PT/Rehab Function

16. Imaging Function

17. Reports Function

18. Sketch Pad Function

19. Documents Function

20. Notes Function

21. Database Function

22. User Function

23. Help Function

24. Macros Function

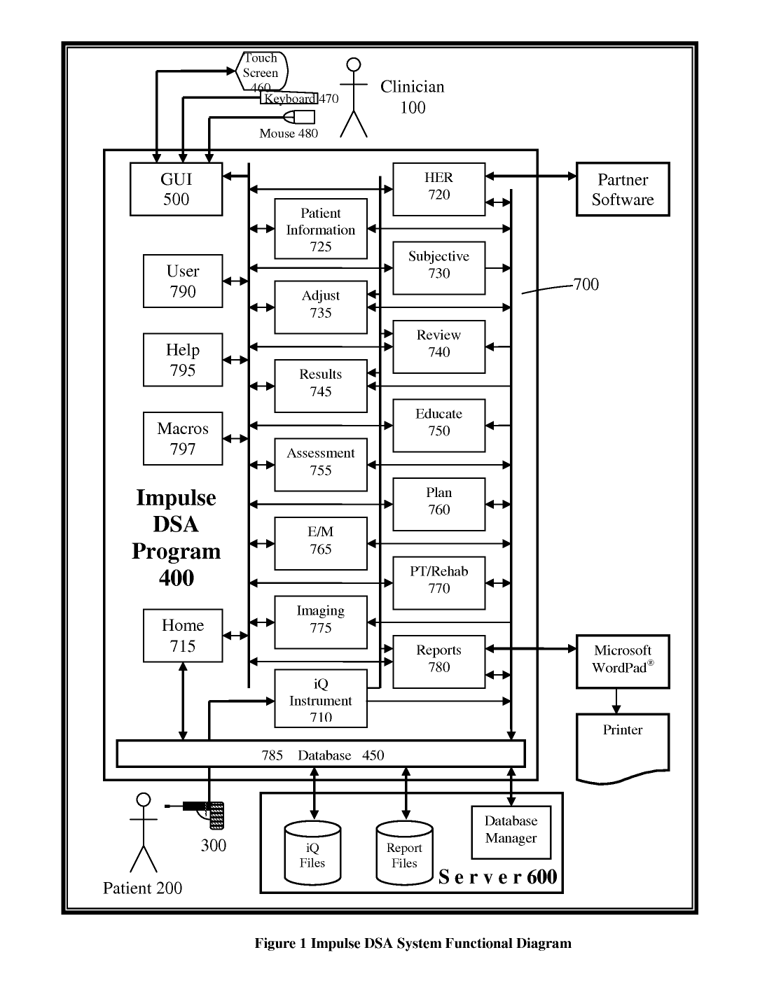

FIG. 1 shows a preferred embodiment where relationships between these functions, the GUI (Graphical User Interface), database, iQ instrument, clinician, and patient. For example, a touch screen is depicted in the figure as part of the system, but is not essential.¹ While FIG. 1 is a preferred embodiment, there may be additional internal interfaces between the functions not shown in FIG. 1. ¹ WordPad is a registered trademark of Microsoft Corporation; but any compatible word processor can be used.

In a preferred embodiment, there are two configurations of the Impulse DSA system may be on a Standalone system where the Impulse DSA program, data files, and database are all resident on a single computer; or a Client-server system where the Impulse DSA program is resident on one or more client workstation computers and the data files and database are hosted on a separate server computer.

FIG. 1 shows a preferred embodiment where Data storage in files and the database via the database manager (DBM) are shown in the Server box. This preferred embodiment represents either the client-server configuration with a remote server or the standalone configuration with a local server resident on the Impulse DSA program host computer. Either configuration may or may not interface with a demographic partner software program.

In a preferred embodiment, most of the functions interface with the GUI and supply it with a window or page that the GUI displays. This may facilitates modular updates to the system. For example, the operator may display pages by clicking or touching buttons or tabs.

In a preferred embodiment, the iQ Instrument Function includes the iQ instrument hardware and firmware and the iQ Instrument software in the Impulse DSA Program that accepts data from the Impulse iQ instrument, stores it to persistent media, and stores a reference to the file in the database. The iQ Instrument software function also reads and formats the iQ data from the iQ instrument and database for other functions.

In a preferred embodiment, the Home Function supplies the Home page to the GUI that is displayed immediately after the operator logs on. The Home page displays the client's clinic logo, name, and any other text demographics the client desires. The Home function preferably also displays buttons for the operator to click or touch to pick an existing patient or enter a new patient. The new patient button is visible only on a system not connected to the client's demographic partner software. The operator selects a patient in order to access any of the other functions that display a page via the GUI.

In a preferred embodiment, the Electronic Health Record (EHR) Function preferably supplies the EHR page to the GUI that may be displayed by operator selection. The EHR function is preferably responsible for interfacing with the client's demographic partner software and allows the operator to run the partner software in a separate process with the click or touch of a button. The Electronic Health Record (EHR) Function is also responsible for formatting and sending information from the Impulse DSA database to the partner software, and receiving and parsing information from the partner software to store in the Impulse DSA database.

In a preferred embodiment, the Patient Information Function preferably supplies the Patient page to the GUI that may be displayed by operator selection. If the system does not interface with a demographic partner program, the operator may create a new patient by entering the patient's information or may edit existing patient information. If the system does interfaces with a partner program, the operator may not create or edit patient information that is retrieved from the partner program.

In a preferred embodiment, the Health History Function preferably supplies the Health History page to the GUI that may be displayed by the operator selection. Health history information about the patient preferably includes patient complaints, review of systems, medical history, conditions or illnesses, family history, social history, and activities of daily living are quantified. Based on patient complaints, appropriate outcome assessment questionnaires may be recommended to be completed by the patient.

In a preferred embodiment, the Subjective Function preferably supplies the Subjective page to the GUI that may be displayed by operator selection. Subjective information about the patient, i.e., the patient's current complaints, is collected on this page.

In a preferred embodiment, the Adjust Function preferably supplies the IAT Adjust page, Freestyle page and the PING Adjust pages to the GUI that may be displayed by operator selection. The clinician may make treatment selections and enter objective findings on these pages. Treatment can be directed by the program and the clinician during which the iQ Instrument function collects data. Both Adjust pages display skeletal graphics to assist in treatment level selection, and feedback from the iQ instrument is displayed in skeletal and waveform frequency animations.

In a preferred embodiment, the operator may use the IAT (Impulse Adjustment Technique) Adjust page to treat the patient from the clinician's objective findings taught in the IAT technique. The Freestyle page can allow the clinician to perform adjustments that are captured as numerical values (treatment Level 1, Level 2, Level 3, and so on) that can be later edited (populated) to document the specific musculoskeletal level. The operator may use the PING Adjust page to ping or tap the patient's skeletal levels with the iQ instrumentas an intermediate step to determine which levels to select for treatment.

In a preferred embodiment, the Review Function supplies the Review page to the GUI that may be displayed by operator selection. The Review page preferably displays most of the items that are displayed on the Adjust pages for the clinician to review the treatment with the patient.

In a preferred embodiment, the Results Function preferably supplies the Results page to the GUI that may be displayed by operator selection. The Results page shows the patient's improvement over time through a number of graphs.

In a preferred embodiment, the Educate Function preferably supplies the Educate page to the GUI that may be displayed by operator selection. The page offers a set of predefined animations which may automatically load based upon the patient's diagnosis and the levels treated on a particular visit. The page may also provide a list of animations from which the operator may select depending upon the patient's diagnosis and the spinal levels treated to educate the patient about his or her condition. It may also offers an animation of the iQ adjusting instrument to teach the patient about its functionality.

In a preferred embodiment, the Assessment Function preferably supplies the Assessment page to the GUI that may be displayed by operator selection. The Assessment page shows a list of the patient's diagnoses in the order entered by the operator. The operator may append new diagnoses to the list.

In a preferred embodiment, the Plan Function preferably supplies the Plan page to the GUI that may be displayed by operator selection. Predefined plan recommendation selections may be available on this page. The clinician may choose from these and make plans for the patient regarding prognosis, treatment, treatment goals, visit frequency, work status, home duties and restrictions, and recommendations.

In a preferred embodiment, the Outcome Measures Function preferably supplies the Outcome Measures page to the GUI that may be displayed by operator selection. Predefined outcome assessment questionnaires are available on this page that are prompted based upon the health history inputs. Upon selection, the patient may be presented with questions to answer regarding their condition and its affect on their overall activities as a means to quantify health status in general and for specific symptomatic presentations. The Outcome Measures results may also be summarized on this page as well as in the Results screen and Adjust Screen.

In a preferred embodiment, the E/M Function preferably supplies the E/M page to the GUI that may be displayed by operator selection. Predefined examination findings selections are available on the E/M page.

In a preferred embodiment, the PT/Rehab Function preferably supplies the PT/Rehab page to the GUI that may be displayed by operator selection. Predefined exercise prescription selections are available for in office and home care.

In a preferred embodiment, the Imaging Function preferably supplies the Imaging page to the GUI that may be displayed by operator selection. The clinician may view patient images; for example, x-rays, for study.

In a preferred embodiment, the Reports Function preferably supplies the Reports page to the GUI that may be displayed by operator selection. The page provides easy control for the operator to view chronologically ordered visit reports for a selected patient. The Reports Function allows the operator to send the current report to the WordPad editor, which can be automatically spawned. This function preferably contains a report generator that is invoked when the patient visit terminates.

In a preferred embodiment, the Sketch Pad Function preferably supplies the Sketch Pad page to the GUI that may be displayed by operator selection. The page provides the ability for the operator to draw on a selection of images on the screen and save the drawings for later retrieval or emailing to the patient.

In a preferred embodiment, the Documents Function preferably supplies the Documents page to the GUI that may be displayed by operator selection enabling a review of saved documents that are organized in the patient's file. X-Ray or MRI reports, health records from elsewhere, and internal documents such as email correspondence or a return to work authorization document, for example may preferably be viewed here.

In a preferred embodiment, the Notes Function supplies the Notes page to the GUI that may be displayed by operator selection that allows the input of notes into the patient file. These notes may include correspondence or communications with the patient that wouldn't normally be held in a daily treatment record; for example, notes regarding a telephone message left as a recall for a patient may be documented here.

In a preferred embodiment, the Database Function consists of the database, iQ data, and report files and the Database software layer that preferably abstracts the system DBM from other program functions. All Impulse DSA program functions, except the Database function itself, perform database access through the Database software layer frontend. The backend of the Database software layer preferably interfaces with the specific DBM allocated to the system. In a preferred embodiment, the DBM specified for the Impulse DSA system is Microsoft SQL Server®. If a another version of the Impulse DSA system allocates a different DBM, only the backend of the Database software layer need be modified. ® SQL Server is a registered trademark of Microsoft Corporation.

In a preferred embodiment, the User Function preferably provides operator login, operator logout, and operator access to the Impulse DSA system. It also allows the operator to create, edit, and delete authorized users of the system.

In a preferred embodiment, the Help Function preferably provides help to the operator in the use of the entire system. Each Impulse DSA program function provides the Help function with its own help page(s). This facilitates modular updates to the system.

In a preferred embodiment, the Macros Function preferably supplies the Macros management window to the GUI. It supports the macro capability for the Impulse DSA program. It maintains system macros deployed with the system, and it maintains operator created macros.

Preferred System Requirements

In a preferred embodiment the invention would include some or all of the following:

Preferably the System Level User Requirements include: a turnkey system (hardware and software); the product software should also be installable on existing hardware systems running Microsoft Windows operating systems such as Windows 7®, Windows Vista®, or Windows XP®; the operator should be permitted to launch multiple instances of the Impulse DSA program; the Impulse DSA program should execute under a license that should expire after a specified time, e.g., one month, after which the program may not run and the client should have a new license installed via the Internet or by manually entering the proper authorization code; and the product should meet CCHIT requirements, HIPPA requirements and ISO requirements. ® Windows 7, Windows Vista, and Windows XP are registered trademarks of Microsoft Corporation.

Preferably the Hardware Level User Requirements include: product hardware should comprising the Impulse iQ instrument, a USB dongle for communications with the Impulse iQ, and an IBM compatible PC with keyboard, mouse, printer, CD and/or DVD drives, touch screen, microphone, sound system, and connection to the Internet; and Specific hardware requirements, such as graphics card, processor speed, and hard drive capacity, would be determined depending on the specific application.

Preferably the iQ Instrument User Requirements include the following: upon completion of the applied thrusts, the instrument should beep as indicated and send one of three indications to the program: (a) a TBD0 signal indicating that the treatment was not successful. The instrument may not beep; (b) a TBD1 signal indicating that there was a significant increase in peak acceleration response, and the response was maximized for a successful treatment; for example, one beep; or (c) a TBD2 signal indicating that there was a significant increase in peak acceleration response, but the response was not yet maximized prior to 48 thrusts being applied for example, two beeps.

Software Level User Requirements

Preferably the Setup Requirements should: the operator to set the client's clinic name and upload the clinic logo and any other clinic demographics during installation and setup and any time during Impulse DSA program operation; provide the means for the operator to input and edit the client's demographic information, such as clinic name, users, address, telephone numbers, fax numbers, email address, web site, logo picture, and other information specific to the client; query the operator during installation of the Impulse DSA program to locate the partner software used and allow the operator to change the partner software information at any time after installation in case the client changes the partner software used or migrates to a newer version of the one used; and attempt to communicate with the partner software and report its success or failure to the operator.

Preferably the Saved Data and Database Requirements should: consist of several table, which include the patient information, patient visits data, and Impulse iQ data and report file references; be encrypted to protect patient privacy; save the patient information in the database; save the Impulse iQ data in files; contain references to the Impulse iQ data files in the database; save the Adjustment results in the database; save Generated reports in files; contain references to the reports files in the database; and have the means for the user to correct a corrupt database.

Preferably the iQ Instrument Function Requirements should include the following: the PC should accept data from the instrument; the PC should accept a single TRIGGER data item from the instrument as an indication that the clinician wants to skip a vertebra in the selection list or two closely spaced, time sequential TRIGGER data items (double click) to indicate that the clinician wants to go back to the proceeding vertebra, and the time interval that determines a double click should be set by the operator using the preferences menu item, or a default is used; the PC should accept the following signals from the iQ instrument: (a) TBD0—the treatment was not successful, (b) TBD1—the treatment was successful, and (c) TBD2—the treatment was not successful after 48 thrusts; and the iQ instrument should accept commands and status information from the PC.

Preferably the GUI Requirements should include the following: the GUI should take advantage of the touch screen. However, it should be kept in mind that since the product software may be installed on an existing PC without a touch screen, all features and functions should be easily accessible with the mouse and keyboard as well; the GUI should display a title bar including an icon, text, and minimize, maximize or restore, close boxes; the name of the client' clinic, and the currently logged on operator; the GUI should display a menu bar with the items for function and ease of use; the GUI should display a toolbar with tools for function and ease of use; the GUI should display a status bar that indicates the status of the Impulse DSA program, e.g., Ready, Busy, Saving File: <filename>. Specific displayed statuses are for function and ease of use; the GUI should display a Patient Bar that displays the patient's ID, name, the number of visits, the current date and the visit duration which can be populated manually or used for demonstration purposes; the Patient Bar should be visible whenever the GUI is visible; The GUI should display the name of the computer workstation on which it is running; The GUI should display buttons or tabs that the user may click or touch to display the following corresponding overlaid pages: (a) Home; (b) EHR (Electronic Health Record); (c) Patient; (d) Subjective; (e) Adjust IAT, Adjust PING, and Adjust FREESTYLE; (f) Review; (g) Educate; (h) Assessment; (i) Plan; (j) E/M; (k) PT/Rehab; (l) Imaging; (m) Reports; (n) Sketchpad; (o) Documents; and (p) Notes; all pages that display the visit date should allow the operator to expand the date to a calendar. The calendar should highlight previous visit dates in yellow and future visit dates in green; and the GUI should have convenient controls for the operator to save the data gathered by the system. Data should be saved on persistent media (local hard drive or server). Backup capability to CDs or DVDs is available in Windows. Regular backup procedures should be explained and encouraged in the product documentation and help sub system.

Preferably the Home Function Requirements should include the following: the Home page should display User Name and Password fields for the attending user to log in; the Home page should display a Login/Logout button that can change changes its name and color based upon login/logout activity; for example, from Green to Red and from “Login” to “Logout;” the Home page should display an Attending Doctor field that upon logging in provides the name of the user under Attending Doctor form a Username database; the Home page should display the client's clinic name and logo and any other clinic demographics the client desires, such as clinic name, users, address, telephone numbers, fax numbers, email address, website, logo picture, and other information specific to the client; the Home page should display buttons that the operator may click or touch, and the buttons should be labeled, or text displayed next to them, indicating the following tasks that the operator may select from (a) New Patient or (b) Other function and ease of use; the Home page should display a New Patient button on a system that does not have partner software, and the New Patient button should automatically assign the next patient number when patient numbers are sequential or the operator should be asked to enter the new patient number, and the New Patient button may take the user to the Patient screen to enter the New Patient Data; the Home page should assist the operator in choosing a patient; for example using the “Patient Name” field or the “Patient ID” field; an x box (close) button may allow the user to close out of a specific patient and all data is automatically saved and the user is preferably prompted to make sure that they want to close out of that specific patient file; and except for the Home page button or tab, all page buttons or tabs should be disabled or not visible until the operator selects a patient and the operator should be permitted to enter a new patient name, ID, and demographics only on a system without demographic partner software or to enter or a Demo mode.

Preferably the EHR Function Requirements should: communicate with the following patient demographic programs that the client may be using; for example, FutureHealth®, ACOM Health®, Eclipse® Healthcare, and ChiroTouch®; display a button that the operator may click or touch to invoke the partner software program resident on the client's system; be capable of sending and receiving patient information data to and from the partner software program; should satisfy CCHIT and HIPPA requirements necessary to be compliant with partner software programs that have these certifications; and on a system that has partner software, the Impulse DSA database should be considered as a secondary (slave) to the partner database (master) where patient information held in the partner software should be considered the primary source while the same information held in the Impulse DSA database should be considered a copy of the source, and may only change if the source data changes. ® FutureHealth is a registered trademark of Future Health, Inc. ACOM Health is a registered trademark of ACOM Solutions, Inc. Eclipse is registered trademark of MPN Software systems. ChiroTouch may be a registered trademark of another company.

Preferably the Patient Information Function Requirements should include the following: the patient data on the Patient page should consist of a number of categories; the first patient data category should display only patient demographic data. the Impulse DSA program should have the ability for the operator to load a picture of the patient that should be displayed on the Patient page for visual identification of the patient; for example, the patient picture may be useful and could be captured by the computer webcam during a patient visit; the patient data on the Patient page should be displayed in a data form of field names next to their corresponding field data, and can, in correspondence with, be accessed from the partner software program used by the client, and should display: (a) First visit date; (b) Last visit date; (c) X-ray date(s); (d) E/M date; (e) Diagnoses; (f) Visit history; (g) Imaging data (the GUI should allow the operator to load x-ray photos or other imaging files which should appear on the Imaging screen) and (h) Tags—(defined automatically populating or user defined) and (i) other patient information categories such as visit number; on a system which has no partner software the Patient page should allow the operator to enter demographic and other information usually provided by partner software; on a system without partner software the patient data field values on the Patient page should be editable by the operator.; on a system with partner software the patient data field values should not be editable and should be imported from the partner software; the Impulse DSA program should recognize changes to the partner database (new or edited data) and import those changes; and the Patient page should provide the means for the operator to cut data from one patient's database records and paste it to another's. This is intended to remedy the case where the data in question was saved under the wrong patient's name.

Preferably the Health History Function Requirements should include the following
1. The Health History page should be made available to new patient prospects to complete on the first visit or on-line using a personal computer (PC) or tablet PC.
2. The Health History page should indicate the progress of which the Health History Questionnaire is completed.
3. The Health History page should display questions and selection controls for input of the patient's responses.
4. The Health History page should display a Next button at the bottom right part of the screen to allow the user to progress to the next page. Progression to the next page may trigger the status bar indicating completion of the questionnaire. The Subjective page should display a Complaints Section containing a full body male or female skeleton picture displaying the skin, depending upon the patient's gender, on which the operator may click or touch a body region. For example, Head/Spine; Lower Back; Ribs; Upper Extremities including Shoulder, Arm, Elbow, Forearm, Wrist, and Hand; and Lower Extremities including Hip, Buttock, Thigh, Knee and Leg/Calf, Ankle, and Foot.
5. The Health History page should display a date field inquiring, “When your complaints begin” for each body region.
6. The Health History page should display the following selectable Symptoms for each body region selection such as Pain, Burning, Dull, Sharp, Shooting, Aching, Throbbing, Numbness, Tingling, Stiffness, Soreness, Swelling and Weakness
7. The Health History page should display the following selectable Intensity ratings for symptoms for each body region selection such as Mild, Moderate and Severe.
8. The Health History page should display the following selectable Timings for symptoms for each body region selection such as Occasional, Intermittent, Frequent and Constant.
9. The Health History page should display the following selectable Responses to treatment of symptoms for each body region selection such as Improving, Worsening, Unchanged and Resolved
10. The Health History page should display a selectable Pain Rating from 0 (best) to 10 (worst).
11. The Health History page should display the following selectable times when symptoms are worse such as Morning, Afternoon, Evening, Night or Other—operator enters when symptoms are worse.
12. The Health History page should display questions and opportunities for responses under the Patient Complaint's section what are the complaints, how did complaint begin and what happened to cause the complaint such as specific event, injury or accident, how would you rate your pain from 0-no pain to 10-worst possible pain, what makes you symptoms better or worse.
13. Have you had any recent treatment for this condition outside of this office? If yes, from whom and for what conditions?
14. Do you suffer headaches? If Yes, describe where the headache is, when the headaches began, the quality of the pain, how often the headaches occur, any associated triggers and how long they last and any other symptoms.
15. The Health History Page should display a Review of Systems section.
16. The Health History Page should display a Social History section.
17. The Health History Page should display a Family History section.
18. The Health History Page should display a Medical History section.
19. The Health History Page should display a Conditions or Illnesses History section.
20. The Health History Page should display a Occupational/Activities History section.
21. The Health History Page should display a Patient signature field.
22. The Following Outcome Assessment Questionnaires should be prompted to be recommended based upon the patient complaints: (a) Neck Disability Index Questionnaire for Neck Pain; (b) Oswestry Low Back Disability Index Questionnaire for Low back Pain; and (c) headache Questionnaire for headaches.

Preferably the Subjective Function Requirements include the following
1. The Subjective page should display buttons and selection controls for input of the patient's subjective condition, i.e., the patient's complaints.
2. The Subjective page should display a full body male or female skeleton picture, depending upon the patient's gender, on which the operator may click or touch a body region; for example, body regions that should be available for selection include: (a) Head/Spine including Head, Neck, Upper Back, Lower Back and Ribs; (b) Upper Extremities including Shoulder, Arm, Elbow, Forearm, Wrist and Hand; and (c) Lower Extremities including Hip, Buttock, Thigh, Knee, Leg/Calf, Ankle, and Foot.
3. The Subjective page should display selectable Symptoms for each body region selection; for example: (a) Pain including Burning, Dull, Sharp, Shooting, Aching, and Throbbing; (b) Numbness; (c) Tingling; (d) Stiffness; (e) Soreness; (f) Swelling; and (g) Weakness
4. The Subjective page should display selectable Intensity ratings for symptoms for each body region selection; for example: Mild, Moderate, and Severe.
5. The Subjective page should display the following selectable Timings for symptoms for each body region selection; for example: Occasional, Intermittent, Frequent, and Constant.
6. The Subjective page should display selectable Responses to treatment of symptoms for each body region selection; for example: Improving, Worsening, Unchanged, and Resolved.
7. The Subjective page should display a selectable Pain Rating from 0 (best) to 10 (worst).
8. The Subjective page should display selectable times when symptoms are worse; for example: Morning, Afternoon, Evening, Night, and Other (operator enters when symptoms are worse).

Preferably the Adjust Function Requirements include the following
1. The Adjust page should consist of the following overlaid pages that the user may select by clicking or touching buttons or tabs: (a) PING (Ping Analysis and Adjustment), (b) IAT (Impulse Adjust Technique), and (c) FREE (Freestyle Adjusting)
2. When the operator clicks or touches the Adjust page, the GUI should assume a new visit for the currently selected patient unless it is on the same day as the previous visit in which case the GUI should ask the operator whether he/she wants to start a new visit for the patient or append the preceding visit.
3. When the operator clicks or touches the Adjust page after selecting a new or existing patient, the Adjust page should be blank.
4. The Adjust pages should display a Last button which the operator may click or touch to populate the page with the last visit's Treatment Data (Level and Side) and the corresponding levels selected on the body image. The user may add or subtract to their selections in this manner. The Last button should be disabled after treatment has begun.
5. The Adjust pages should display a Clear button which the operator may click or touch to clear the last visit data. The Clear button should be disabled after treatment has begun.
6. The Adjust pages should display a skeleton picture of the current patient's gender initially as full, back view with transparent skin overlay (default). The height of the skeleton picture should be as large as is permitted by the height of the GUI window minus its title, menu, status, and Patient bar heights. The width of the skeleton picture should be that required by the skeleton picture proportions.
7. The Adjust pages should display a rotate button to rotate the skeleton picture between back, right, front and left views. This rotation capability may be capable in the default full body view and in the Level 1 and Level 2 zoom views defined below. The Adjust pages should display a muscles button to provide an overlay of selectable muscles to which treatment may be applied. This functionality should be available in the full body view and each of the Level 1 and Level 2 zoom views.
8. When the operator hovers over an available selectable area (defined as a hotspot) transparent grey shading should provide the user with feedback over which level is currently qued to be selected should the user click or touch the screen. This hovering capability should exist for the Full Body View and Level 1 and Level 2 Zoom views.
9. From the Full Body View, when the operator touches a selectable region of the skeleton picture, the graphics should animate a zoom to that region. Level 1 and Level 2 Zoom views are provided: (a) Head/Spine/Pelvis including (i) Head and Cervical Spine, (ii) Thoracic Spine and Ribs, and (iii) Lumbar Spine and Pelvis; (b) Upper Extremities including (i) Shoulder left and right, (ii) Elbow left and right and (iii) Wrist and Hand left and right; and (c) Lower Extremities including (i) Hip-left and right, (ii) Knee left and right and (iii) Ankle and Foot left and right.
10. Level 1 Zoom should be made possible by touching or clicking anywhere in the corresponding body regions: (a) Head, Spine, and Pelvis; (b) Left Upper Extremity; (c0 Right Upper Extremity; (d) Left Lower Extremity; and (e) Right Lower Extremity.
11. Level 1 and Level 2 Zoom should provide hotspots for treatment selections.
12. Zoom navigational buttons should be included in the white space surrounding the body image for the Full Body View and Level 1 (+) and Level 2 (++) zooms enabling the operator to navigate the zoom functionality to a particular body region. Zooming out should be accomplished by clicking or touching anywhere in the white space or by touching the “−” button.
13. The Adjust page should display a Selection List in which is placed information for the treatment of the patient.
14. When the operator clicks or touches a selectable skeletal part in a Level 1 or Level 2 zoomed region, the selected part in the zoomed skeletal picture may turn red and the name of that part should be appended to the Treatment Selection list. By clicking or touching the same selection, the operator may toggle the selection into and out of the Selection List and the skeleton graphics between red and its original color.
15. Based upon the selection by the operator the side (left, right, or bilateral) may be captured and indicated in the Treatment Selection List.
16. Selectable skeletal parts for each region may be those listed on the List sheet in Impulse DSA Database Pick List.
17. The Selection List on the Adjust page should display columns with the following headings: (a) Treatment including (i) Level—the skeleton level or muscle treated and (ii) Side—the side treated; (b) Objective PART Findings in the region including (i) Pain—tenderness; (ii) Asymm—asymmetry noted, (iii) ROM—Restriction in range of Motion noted, and (iv) Tone—The texture, tone, or myospasm noted; (c) Mobility including (i) Pre—first hit value, (ii) Post—last hit value, (iii) % Imp—percent improvement in acceleration amplitude or derived displacement from the initial thrust to the final thrust determined by the following calculation, and (d) Thrusts—Appended number of thrusts administered during treatment to the selected level.
18. The Selection List may allow the operator to drag and drop a level row in the list to change the order of that level in the list.
19. The Selection List should provide the means for the operator to delete level row entries from the list. This may be done by the operator highlighting a row and pressing the delete key.
20. The Selection List should show an indication, e.g., a checkmark or letter, in the Pain, Asymm, ROM, Myosp, and Inflam columns when the operator clicks or touches that column in a level row entry. These are called objective findings.
21. A second level of objective findings to provide qualifiers and more detail into the patient record should be made available by clicking or touching the Details button in the Objective PART Findings window.
22. The Selection List should display data received from the iQ instrument in the Pre Mobility, Post Mobility, and Thrusts columns for each level treated in a bar graph display. The Pre Mobility is the acceleration value (m/s²) of the first instrument thrust can be a first bar and the Post Mobility is the acceleration value of the last instrument thrust displayed in a second bar. Colors corresponding to the real-time frequency graphic display of red, yellow and green can be defined and displayed in these results.
23. A Ready button should provide functionality to inform the GUI that the operator is ready to begin treatment. Clicking or touching the ready button from any level zoom may bring the window to the “Back” Full Body view where all selected levels should be visualized.
24. When the Ready button is clicked or touched, the first selection in the Treatment Menu should change color and background highlighted to indicate the level of selection being treated. On the Full Body View, crosshairs may appear over the selection to indicate where treatment is to be applied.
25. A Done button should provide functionality to inform the GUI that the treatment is finished and all data may be automatically saved upon touching or clicking this button.
26. The Adjust pages should accept a single click from the instrument that indicates that treatment has begun.
27. The Adjust pages should display the Current Level window that should display the current level to be treated from the Selection List. The Current Level window should display text that is large enough to be clearly seen from the treatment location, but no more than ten feet away.
28. The Adjust pages should display a Thrust Count window that should display the number of thrusts in real time being delivered to the selected level. This numerical value should append until the end of the treatment to the selected level. This value should then be captured under “Thrusts” in the Treatment menu and then reset to zero for the next selection.
29. The skeleton picture level corresponding to the level displayed in the Current Level window should blink (pulsate) when selected during treatment. Crosshairs should indicate the current level being treated.
30. The system should announce (preferably audibly) the current level to be treated when treatment begins and when the level displayed in the Current Level window changes to the next level in the Selection List. This functionality should also be able to be turned off under user preferences.
31. The Adjust pages may display a button that the operator may click or touch to toggle the muting of the treatment level announcements
32. The skeleton picture should show the level treated in green when the treatment is successfully completed.
33. When the current treatment selection is completed, the next level in the selection list should become the current level and be displayed in the Current Level window. Likewise, the next selection may blink (pulsate) and crosshairs may appear indicating the specific contact for treatment.
34. During treatment, if the treatment is not successful, the level selected should turn yellow and the selected level may remain selected to append data for continued treatment.
35. The GUI should accept a command from the operator to change the current level by mouse click, touch screen, or voice prompt.
36. Upon the operator single clicking the instrument, the Adjust page should display the next level to be treated from the Selection List in the Current Level window, highlight the level in the Selection List, announce the level, and blink the skeletal level and place crosshairs in the skeleton picture.
37. Selectable crosshair icons may be made available under user preferences.
38. Upon the operator double clicking the instrument, the Adjust page should display the previous level to be treated from the Selection List in the Current Level window, highlight the level in the Selection List, announce the level, blink the skeletal level and place crosshairs in the skeleton picture (Data may be appended or overwritten during this feature).
39. Upon completion of the treatment of a level, the results of the treatment should be displayed in the Selection List Mobility Pre, Mobility Post, and % Imp columns for that level.
40. Upon completion of the applied thrusts, the instrument should beep as indicated and send one of three indications to the program:
   a. A TBD0 signal indicating that the treatment was not successful. The instrument may not beep.
   b. A TBD1 signal indicating that there was a significant increase in peak acceleration response, and the response was maximized for a successful treatment. The instrument beeps once.
   c. A TBD2 signal indicating that there was a significant increase in peak acceleration response, but the response was not yet maximized prior to 48 thrusts being applied and thus the treatment is considered not successful. The instrument beeps twice.
41. Upon reception of the TBD0 signal from the instrument, the IAT Adjust page shall
   a. not change to the next level in the Selection List anticipating continued thrusting on the current level,
   b. record and display the pre-post result, i.e., % improvement calculation, in the Selection List % Imp (Biom Result) column,
   c. change the color of the level in the skeleton picture to yellow and continue blinking with crosshairs remaining in the same location (note that the operator may accept this outcome by advancing to the next level by single clicking the instrument in which case the level in the skeleton picture should remain yellow without blinking),
   d. record the next pulse train for this level overwriting the previous results, but add to the thrust count.
42. Upon reception of the TBD1 sig-nal from the instrument, the IAT Adjust page shall
   a. record and display the pre-post result, i.e., % improvement calculation, in the Selection List % Imp (Biom Result) column,
   b. record the number of thrusts for the level in the Thrust Count column,
   c. turn the skeletal level green,
   d. highlight the next level in the Selection List, display that level in the Current Level window, announce that level, and blink that level with crosshairs in place in the skeleton picture.
43. Upon reception of the TBD2 signal from the instrument, the IAT Adjust page shall
   a. record and display the pre-post result, i.e., % improvement calculation, in the Selection List % Imp (Biom Result) column,
   b. change the color of the level in the skeleton picture to yellow and continue blinking (note that the operator may accept this outcome by advancing to the next level by single clicking the instrument in which case the level in the skeleton picture should turn green without blinking),
   c. record the next pulse train for this level appending the previous results, and adding to the thrust count.
44. When the operator has indicated the treatment of a previously treated level by the operator double and single clicking the instrument to move to the desired level, the Adjust pages should overwrite the previous results with the new results received from the instrument.
45. In the skeleton picture, preferably, a red colored level should indicate that it is in the Selection list and is to be treated, a green colored level should indicate that it has been treated successfully, a yellow colored level should indicate that it has been treated unsuccessfully, and its original color should indicate that it is not to be treated.
46. The Adjust pages should have a Done button that should close the current treatment session.
47. When the operator clicks or touches the Done button on the Adjust pages, the Impulse DSA program should export the collected patient data to the partner software.
48. The operator may run as many instances of the Impulse DSA program as desired. In this way the operator may treat several patients without submitting their data, and may finish the documentation for their visits and submit them at a later time.
49. The operator may open a patient's treatment data at a later time to complete and/or modify that patient's treatment documentation, and then submit it.
50. The operator may open a patient's treatment data at a later time to readjust a level that has already been selected. In this case the Impulse DSA program should discard the previous data and overwrite it.
51. The operator may open a patient's treatment data at a later time to adjust a level that was not originally selected. In this case the Impulse DSA program should append the new treatment data to the previous data.
52. The IAT Adjust page should allow the operator to select from a list of macros called Protocols. The following protocol buttons should be defined: (a) Headache, (b) Cerv-Brach, (c) Rot. Cuff, (d) Ant. Should, (e) AC Joint, (f) Lat. Epi., (g) Med. Epi., (h) CT Synd., (i) Wrist, (j) SIJ/IR, (k) SIJ/ER, (l) Post Knee, (m) Ant Knee, (n) Med. Knee, (o) Lat. Knee, (p) Plant. Fasc., (q) Ach. Tend., (r) Inv. Ankle, (s) Ev. Ankle, (t) Metatarsals.
53. Left, Bilateral, and Right Buttons should be visible on the GUI under the Protocols section to enable the operator to define which side they wish to select the specific protocol for.
54. When a Protocol is selected the Adjust page should place its expansion, i.e., list of levels, in the Selection list and color the levels red in the skeleton picture. The defined levels of the Protocols are provided in the Impulse DSA Pick List/Protocols Worksheet of the files.
55. The Adjust page should display the Thrust Count window that should display the number of thrusts administered during the treatment on a given visit (4 digits available).
56. The Adjust page should display the Visit Duration in a Timer window that should display the time from the beginning of treatment when the IAT Adjust page was selected to the end of treatment when the Donebutton was clicked or touched.
57. The Adjust page should display the iQ Waveform Frequency window that should display a rendition of the instrument frequency data in real time during the adjustment.
58. The iQ Waveform Frequency window should display a representation of the amplitude, frequency, and width (time duration) of the pulses delivered by the instrument. An audible representation of the amplitude, frequency, and width of the pulses should also be generated. The audible representation should be able to be turned on or off in user preferences.
59. The Adjust pages should display the Spinal Motion window that should display a rendition of the instruments vibrations as applied to the skeletal level.
60. The Adjust pages should allow the operator to select whether the iQ Waveform Frequency window or the Spinal Motion window to be displayed. The iQ Waveform Frequency window should be displayed as the default.
61. The Adjust pages should display the Frequency Meter window that should display a speedometer style meter in real time. The initial thrust frequency should show as a static red needle on the speedometer, while the final thrust frequency should show as a static green needle. A black dynamic needle should show the current thrust frequency during treatment. The Frequency Meter window should also display the frequency as a numerical value.
62. The Adjust pages should display the Visit Results bar graph that should show the pre (first thrust) and post (last thrust) results of the visit. The x-axis should represent the levels from the selection list while the y-axis should represent the mobility improvement pre and post results. The pre results should be shown in red and the post results should be shown in green.
63. The Adjust page should contain three selectable pages: the Impulse Adjusting Technique (IAT) page,the Ping Protocol Diagnostic Mode and Treatment (PING) page, and the Freestyle Page
64. The Freestlye page may act the same as the IAT page, except allow the operator to begin treatment without making any selections on the Body. When the operator touches the Ready button they can begin treatment. Using the same Instrument functionality protocols as IAT, when treatment is initiated a number 1 should appear in the Level field and when a successful treatment is made, upon the next initiation of a treatment, consecutive numbering may appear in the Level section of the Treatment screen.
65. The Freestyle Page should have a Populate button, that when pushed highlights the first Level selection and allows the operator to touch the skeleton body GUI to make the Level selection, for example turning the Number 1 to L5 Left. After each selection is made the system should automatically prompt the next consecutive level until all levels are changed from numerical values to body levels.
66. Should the operator try and close out of the patient file prior to populating the Levels list or if the levels list is not fully populated, the GUI should prompt them before closing.
67. The PING Adjust page should display the same controls and features as the IAT Adjust page except as explicitly noted in the user requirements.
68. The PING Adjust page should record and display the instrument data as the operator pings (taps) the patient's skeletal levels with the instrument once on each selected level.
69. The PING Adjust page should not display selectable macros.
70. The PING Adjust page should not display objective findings, i.e., Pain, Assym, ROM, Myo, and Inf, in the Selection List.
71. The PING Adjust page should display the Body Area List from which the operator may select by clicking or touching the following list items:
   a. Full Spine C0-S1
   b. Cervical Cervical Spine: C0-C7
   c. Thoracic Thoracic Spine: T1-T12
   d. Lumbosacral Lumbar Spine and Sacrum: L1-S1
72. The PING Adjust page should display the Analyze Queue with columns Left, Level, and Right. The Left column should display the pre mobility (first hit acceleration value from the instrument) for the left tap on the level. The Right column should display the pre mobility value from the right tap on the level. The Level column should display the pre mobility value from the center tap on the level.
73. The PING Adjust page may display the Analyze PA button which the operator may click or touch to indicate posteroanterior motion response of the spine comparing level to level.
74. The PING Adjust page may display the Analyze AX button which the operator may click or touch to indicate axial motion response of the spine comparing right to left sides of each level.
75. The PING Adjust page may append left to right upper extremity macros into the Analyze Queue when the operator clicks or touches Upper Extremity in the Body Area List.
76. The PING Adjust page may append left to right lower extremity macros into the Analyze Queue when the operator clicks or touches Lower Extremity in the Body Area List
77. The PING Adjust page should display a Ready button that the operator may click or touch to begin taps on the patient. When the operator clicks or touches the Ready button, the system should anticipate receiving instrument data for the first level in the Analyze Queue.
78. Upon receiving instrument data from the current single thrust, the PING Adjust page should emit a single beep and display the mobility pre results in the Analyze Queue next to the pinged current level. The system should then anticipate receiving instrument data for the next level in the Analyze Queue.
79. When the PING Adjust page receives instrument data for the last level in the Analyze Queue, it should emit a double beep and display the mobility pre results in the Analyze Queue.
80. At the completion of the ping sequence in the Analyze Queue, the PING Adjust page should display the columns in the Analyze Queue according to their mobility value. The stiffest levels should have their rows in the Analyze Queue colored red, less stiff yellow, and least stiff green. The thresholds for each mobility range should be user configurable during installation of the Impulse DSA program and on the options menu.
81. The PING Adjust page should place levels in the Analyze Queue that are clicked or touched by the operator into the Selection List.
82. As in the IAT Adjust page, the PING Adjust page should allow the operator to select any levels to be treated by clicking or touching them in the skeleton picture. Selected levels should be appended to the Selection List.

Preferably the Review Function Requirements should include the following:
1. Data from the patient's last visit should initially be displayed on the Review page.
2. The patient information displayed on the Review page should consist of the levels treated, each level's objective findings, pre and post mobility and percent improvement results, and the thrust count.
3. The Review page should display forward and back arrow buttons which the operator may click or touch to display patient information from the previous (back arrow) or next (forward arrow) visit.
4. The Review page should allow the operator to enlarge the iQ Waveform Frequency window for patient education.
5. The Review page should allow the operator to select whether the iQ Waveform Frequency window or the Spinal Motion window is to be displayed. The iQ Waveform Frequency window should be displayed as the default.
6. The Review page should allow the operator to select the skeletal level to be displayed for playback on the iQ Waveform Frequency window and the Spinal Motion window.

Preferably the Results Function Requirements should include the following:
1. The Results page should show the patient's improvement over time with graphical representations of the visit data.
2. A Results page should display the cumulative results of an adjustment, accumulated from a set of selected visits. The display should be a three-axis graph showing the amplitude and frequency of the pulses as a function of time.
3. The Results page should graphically show the number of levels needing treatment in the Number of Levels graph. The Number of Levels graph x-axis should represent the visit number while the y-axis should represent the total number of levels treated on the visit. A bar graph with trend line should be used.
4. The Results page should graphically show the number of thrusts delivered by summing the thrust count in the Number of Thrusts graph. The Number of Thrusts graph x-axis should represent the visit number while the y-axis should represent the total number of thrusts delivered on that visit. A bar graph with trend line should be used.
5. The Results page should graphically show the mean improvement in mobility for the levels treated in the Mobility graph. The Mobility graph x-axis should represent the visit number while the y-axis should represent the average mobility for that visit. A bar graph with trend line should be used.
6. The Results page should graphically show the mean stiffness index for the levels treated in the Stiffness Index graph. The Stiffness Index graph x-axis should represent the visit number while the y-axis should represent the average stiffness index for that visit. A bar graph with trend line should be used.
7. The Results page should graphically show the mean improvement in frequency for the levels treated in the Frequency graph. The Frequency graph x-axis should represent the visit number while the y-axis should represent the average frequency for that visit. A bar graph with trend line should be used.
8. The Results page may graphically show the mean improvement in Thrust count for the number of visits that the patient has had plotting thrust count in the y-axis and the visit number on the x-axis.
9. The Results page should graphically show the mean improvement in pain (VAS) for the levels treated in the Pain graph. The Pain graph x-axis should represent the visit number while the y-axis should represent the VAS score from Subjective page for that visit. A bar graph with trend line should be used.

Preferably the Educate Function Requirements should: display a list of predefined canned animations that are related to the patient's diagnoses and levels treated and the operator may click or touch items in the list to play the animations; and display a button to play a predefined animation of the adjusting instrument for the patient's education about its functionality.

Preferably the Assessment Function Requirements should: list the patient's diagnoses in numerical or chronological order; allow the operator to add diagnoses; and allow the operator to select from the following predefined progress reports: (a) Percent improvement of spasm, (b) Percent improvement of tenderness, (c) Percent improvement of inflammation, (d) Percent improvement of range of motion, and (e) Percent improvement of activities of daily living

Preferably the Plan Function Requirements should include the following: The Plan page should allow the operator to select from the following list of predefined recommendations: (a) Treatments including (i) Manual therapies, (ii) Physical modalities, and (iii) Rehabilitation; (b) Treatment goals; (c) Visit frequency; (d) Work status; (e) Home duties and restrictions; (f) Recommendations; and (g) Prognosis.

Preferably the Outcome Assessment Function Requirements should include the following: the Following Outcome Assessment Questionnaires may be prompted to be recommended based upon the patient complaints: (a) Neck Pain—Neck Disability Index Questionnaire, (b) Low Back Pain—Oswestry Low Back Disability Index Questionnaire, (c) Headaches—Headache Questionnaire, and (d) Others Questionnaires.

Preferably the E/M Function Requirements should include the following: the E/M page should allow the operator to report predefined examination findings.

Preferably the PT/Rehab Function Requirements should include the following: the PT/Rehab page should display a predefined list of exercise prescriptions for in-office and home care from which the operator may choose for the patient.

Preferably the Imaging Function Requirements Requirements should include the following: the Imaging page should allow the operator to view x-ray and other images of the patient.

Preferably the Reports Function Requirements should include the following: the GUI should provide means for the operator to generate patient reports. The reports should display a summary of each visit: spinal level, right or left side, pre-post result, etc. The reports should be scrollable by touching a back arrow or forward arrow to scroll between visits. Alternatively, a graph of improvements should be displayed above the reports window in which the reports should scroll when the operator touches and slides on the graph; the Reports page should allow the operator to print reports; the Reports page should allow the operator to edit reports; the Reports page should display automatically generated reports for each patient visit; and the Reports page should display back and forward arrow buttons which the operator may click or touch to view the previous or next visit report, respectively.

Preferably the Database Function Requirements should include the following: the Database function should allow the operator to archive patient data files and database entries to CD or DVD; the Database function should allow the operator to retrieve patient data files and database entries from CD or DVD archives; and the Database function should provide screens for the operator to manage the database. Database management systems have such user interfaces, but are usually designed for a database technician.

Preferably the User Function Requirements should include the following: the User function should require the operator to login with a user ID and password, and the DSA program should not run unless the entered user ID and password are valid; and the User function should allow the operator to set valid user ID and password pairs, and the valid user ID and password pairs should be encrypted and stored on persistent media.

Preferably the Help Function Requirements should include the following (1) The Help function should be accessible from any screen of the system during the operation of the product, and the help subsystem should consist of a set HTML files and/or a single CHM file; (2) the help files should include text and graphics to help the user understand and use the system efficiently and productively, and the granularity of the help provided by the help subsystem should be to the extent that it covers every function and feature of the product and ensures that the user may have a complete understanding of its full capabilities; (3) Context sensitive help; and (4) Each software function should provide the Help function with help pages that explain the function and its capabilities accessible to the operator.

Preferably the Macros (Protocols) Function Requirements should include the following: the Impulse DSA program should provide predefined macros with body regions. Each set of macros associated with a body region should define a macro category; the Impulse DSA program should allow the operator to define macros and assign them to macro category; and the Impulse DSA program should allow the operator to create, edit, and delete macros. Preferably, the macros included during program installation may not be edited or deleted by the operator.

Software Requirements

This section presents some preferred Impulse DSA software requirements.

TABLE 1
System Requirements Traceability Referencing

| Prefix | System Requirements Group |
|---|---|
| CON | Constraints |
| HWR | Hardware Related Requirements |
| IQI | iQ Instrument Requirements |
| GEN | General Requirements |
| GUI | Graphical User Interface (GUI) Requirements |
| HOM | Home Page Requirements |
| EHR | EHR Function Requirements |
| PAT | Patient Information Function Requirements |
| SUB | Subjective Function Requirements |
| ADJ | Adjust Function Requirements |
| REV | Review Function Requirements |
| RES | Results Function Requirements |
| EDU | Educate Function Requirements |
| ASM | Assessment Function Requirements |
| PLN | Plan Function Requirements |
| EMF | E/M Function Requirements |
| PTR | PT/Rehab Function Requirements |
| IMG | Imaging Function Requirements |
| RPT | Reports Function Requirements |
| DBF | Database Function Requirements |
| USR | User Function Requirements |
| HLP | Help Function Requirements |
| MAC | Macro Requirements |

Interface Requirements

Preferably the External Interfaces should include the following: Operator Interface—The Impulse DSA Program should interface with the operator via the Graphical User Interface (GUI); iQ Instrument Interface—The Impulse DSA Program should interface with the iQ instrument; Editor Program—The Impulse DSA Program interfaces with a resident editor on the host computer; and the operator may edit and print reports with the edit program; and External Systems Interface—The Impulse DSA Program should interface with the client's demographic partner software programs via the Internet, local intranet, or local pipe.

Internal Interfaces

Table 2 lists a preferred embodiment of internal interfaces depicted in FIG. 1 and gives an indication of the information passed between the software functions and the GUI. Preferable Internal interface requirements are presented within the appropriate software function subsections in the present section.

TABLE 2
Software Internal Interfaces

| | Interfaces | |
|---|---|---|
| Function | With . . . | To . . . |
| iQ Instrument | Adjust | Provide iQ instrument data |
| | Review | |
| | Results | |
| | Reports | |
| | Database | Store iQ instrument data file reference |
| Home | GUI | Display Home page |
| | | Choose patient |
| | Database | Get and set patient information |
| EHR | GUI | Display EHR page |
| | Partner S/W | Spawn demographic partner software |
| | | Get patient demographic information |
| | Database | Store patient demographic information |
| Patient Information | GUI | Display Patient page |
| | | Display and edit patient information |
| | Database | Get and set patient information |
| Subjective | GUI | Display Subjective page |
| | Database | Store patient subjective data |
| Adjust | GUI | Display Adjust page |
| | | Display skeleton picture with zoom selection |
| | | Display and edit selection list |
| | | Display macro selection list |
| | | Display and edit objective findings |
| | | Display iQ data as waveform and level vibrations |
| | | Display calculated results . . . |
| | Database | Get and set last visit selection list, etc. |
| | iQ Instrument | Get and display iQ data |
| Review | GUI | Display Review page |
| | Database | Get and set last visit selection list, etc. |
| | iQ Instrument | Get and display iQ data |
| Results | GUI | Display Results page |
| | Database | Get visit data for display |
| | iQ Instrument | Get and display iQ data |
| Educate | GUI | Display Educate page and canned animations |
| | Database | Get patient subjective and objective findings |
| Assessment | GUI | Display Assessment page |
| | Database | Get patient's diagnoses for view and edit |
| Plan | GUI | Display Plan page |
| | Database | Get and set plan recommendations |
| E/M | GUI | Display E/M page |
| | Database | Get and set examination findings |
| PT/Rehab | GUI | Display PT/Rehab page |
| | Database | Get and set patient prescriptions |
| Imaging | GUI | Display Imaging page |
| | | Display patient images |
| Reports | GUI | Display Reports page |
| | Database | Generate reports |
| | | Get, view, edit, and set report files |
| Database | GUI | Database Management |
| | All functions | Get and set data |
| User | GUI | Provide logon window |
| | | Get and set authorized users |
| Help | GUI | Display help pages |
| | All functions | Get help pages |

Functional Requirements

The following subsections present the requirements for the preferred software function described in preferred functions. For each software function the preferred requirements are categorized as: (1) System Requirements—The system requirements are included for traceability to each of the software functions; (2) Software Requirements; (3) Functional Requirements—Functional and behavioral requirement; (4) Data Requirements—Requirements for the maintenance and manipulation of data item; (5) Internal Interface Requirements—Requirements for interfacing with other software functions and the GUI; and (6) External Interface Requirements—Requirements for interfacing with objects external to the software program

In the current preferred embodiment of the invention in FIG. 1, a clinician 100 works with a patient 200 to treat skeletal maladies. The clinician 100 works with an electric chiropractic adjusting instrument 300 and software program 400 to monitor the patient's progress and modify the adjusting instrument 300. The software program 400 gathers, records, and analyzes data provided by the adjusting instrument 300 to give the clinician 100 real time results regarding the patient's body with respect to the adjustment procedure. This allows the clinician 100 to change his or her adjustment techniques as well as the chiropractic adjusting instrument 300 based on the information.

The clinician 100 begins by adjusting the spine of a patient 100 with an electric chiropractic adjusting instrument 300. As the clinician 100 adjusts the patient's spine, the chiropractic adjusting instrument 300 receives multiple data points from the patient 200. These data points include the chiropractic adjusting instrument 300 thrust data, the patient 200 responses to the thrusts of the chiropractic adjusting instrument 300, and the objective clinician 100 findings. All of these data points are transmitted and recorded in a database 450. This database of information is then used with the software program 400 to provide different information based on performed functions to the clinician 100. In a preferred embodiment, the database 450 is stored locally on a single computer. In an alternate embodiment, the software program 400 is resident on one or more client workstation computers and the database 450 is hosted on a separate server computer 600.

Through a graphical user interface (GUI) 500, the clinician 100 can direct the software program 400 to interpret and display the data in multiple ways. In a preferred embodiment, the clinician 100 manipulates the software program 400 through a touch screen 460, a keyboard 470, and a mouse 480. The clinician 100 can generate reports through the software 400 based on the recorded data, and further produce graphs and other output to plan further treatment of a patient 200. In the same way, the clinician 100 can modify the settings on the chiropractic adjusting instrument 300 based on the results presented by the software from the patient 200 during treatment.

The software program 400 includes several functions 700 that can be used to manipulate the data to provide the clinician 100 with valuable results relating to the patient 200. In particular, the functions include the electric chiropractic adjusting instrument function 710, the home function 715, the electronic health record function 720, the patient information function 725, the subjective function 730, the adjust function 735, the review function 740, the results function 745, the educate function 750, the assessment function 755, the plan function 760, the E/M (examination findings) function 765, the Physical Therapy/Rehab function 770, the imaging function 775, the reports function 780, the database function 785, the user function 790, the help function 795, and the macros function 797.

What is claimed is:

1. A method for interpreting patient treatment results and data using an electric chiropractic adjusting instrument and software program comprising the steps of:
a. adjusting a spine of a patient using the electric chiropractic adjusting instrument;
b. gathering electric chiropractic instrument adjustment thrust data, patient spine response to thrusts of the electric chiropractic adjustment instrument, and objective clinician findings;
c. recording the electric chiropractic instrument adjustment thrust data, the patient spine response to the thrusts, and the objective clinician findings in a database;
d. providing real time results and findings; and
e. generating reports based on the thrust data, the patient spine response to the thrusts of the electric chiropractic adjustment instrument, and the objective clinician findings.

2. The method of claim 1 further comprising the step of displaying the real time results and findings on a graphical user interface.

3. The method of claim 1 further comprising the step of analyzing and recording subjective findings from the patient.

4. The method of claim 1 further comprising the step of producing graphs for clinician analysis to plan further treatment of the patient based on the results and findings.

5. The method of claim 1 further comprising the step of storing the results and data on a remote server.

6. The method of claim 1 further comprising the step of storing the results and data on a local server.

7. A method to modify an electric chiropractic adjusting instrument thrust adjustment using recorded patient data comprising the steps of:
a. gathering electric chiropractic instrument adjustment thrust data, patient spine response to thrusts of the electric chiropractic adjustment instrument, and objective clinician findings;
b. recording the electric chiropractic instrument adjustment thrust data, the patient spine response to the thrusts, and the objective clinician findings in a database;
c. providing real time results and findings to a clinician based on the gathered data; and
d. modifying the thrust adjustment of the electric chiropractic instrument based on the adjustment thrust data, the patient spine response to the thrusts, and the objective clinician findings.

8. The method of claim 7 further comprising the step of displaying the real time results on a graphical user interface.

9. The method of claim 7 further comprising the step of analyzing and recording subjective findings from the patient.

10. The method of claim 7 further comprising the step of producing graphs for clinician analysis to modify the electric chiropractic adjusting instrument based on the results and findings.
